(19) United States
(12) Patent Application Publication
(10) Pub. No.: US 2004/0105857 A1
(43) Pub. Date: Jun. 3, 2004

(54) Methods and compositions for enhancing angiogenesis

(76) Inventors: Anthony Montgomery, San Diego, CA (US); Peter Brooks, Carmel, NY (US); Ralph Reisfeld, La Jolla, CA (US)

Correspondence Address: Peng Chen, Morrison & Foerster, Suite 500, 3811 Valley Centre Drive, San Diego, CA, 92130-2332, US

(21) Appl. No.: 10/130,337

(22) PCT Filed: Sep. 26, 2001
(86) PCT No.: PCT/US01/42375

(51) Int. Cl. A61K 39/395

(52) U.S. Cl. 424/144.1

(57) ABSTRACT

This invention relates generally to the field of angiogenesis. In particular, the invention provides a method for enhancing angiogenesis, which method comprises administering an effective amount of an integrin binding pro-angiogenic agent or an integrin antagonist to a mammal, wherein angiogenesis is desirable, thereby enhancing angiogenesis in said mammal. The invention also provides isolated, angiogenic proteins or peptides or isolated nucleic acids encoding the angiogenic proteins or peptides. Combinations and methods for enhancing angiogenesis are further provided.

### TECHNICAL FIELD

This invention relates generally to the field of angiogenesis. In particular, the invention provides a method for enhancing angiogenesis, which method comprises administering an effective amount of an integrin binding pro-angiogenic agent or an integrin antagonist to a mammal, wherein angiogenesis is desirable, thereby enhancing angiogenesis in said mammal. The invention also provides isolated, angiogenic proteins or peptides or isolated nucleic acids encoding the angiogenic proteins or peptides. Combinations and methods for enhancing angiogenesis are further provided.

### BACKGROUND ART

Angiogenesis is the generation of new blood vessels from parent microvessels. Angiogenesis is essential for normal placental, embryonic, fetal and post-natal development and growth, but almost never occurs physiologically in adulthood except under strictly controlled conditions, and except cyclically in the ovarian follicle, corpus luteum and post-menstrual endometrium (Norrby, APMIS, 105:417-437 (1997)).

Angiogenesis is highly regulated by a system of angiogenic stimulators and inhibitors. Known examples of angiogenesis stimulators include certain growth factors, cytokines, proteins, peptides, carbohydrates and lipids (Norrby, APMIS, 105:417-437 (1997); Polverini, Crit. Rev. Oral. Biol. Med., 6:230-247 (1995)). A variety of endogenous and exogenous angiogenesis inhibitors are known in the art (Jackson et al., FASEB, 11:457-465 (1997); Norrby, APMIS, 105:417-437 (1997); and O'Reilly, Investigational New Drugs, 15:5-13 (1997)). Certain diseses or disorders, scuh as ischemic diseases or wound healing disorders, are associated with deficient angiogenesis.

Therefore, it is an object of the present invention to provide compositions and methods for enhancing angiogenesis when such angiogenesis is desirable. It is also an object of the present invention to provide compositions and methods for treating diseases or disorders associated with deficient angiogenesis.

### DISCLOSURE OF THE INVENTION

This invention relates generally to the field of angiogenesis. In one aspect, the invention is directed to a method for enhancing angiogenesis, which method comprises administering an effective amount of an integrin binding pro-angiogenic agent to a mammal, wherein angiogenesis is desirable, thereby enhancing angiogenesis in said mammal. The exemplary integrin binding pro-angiogenic agent can be protein, polypeptide or peptide, or small molecule pro-angiogenic agent that contains an integrin binding sequence. For protein, polypeptide or peptide, or small molecule pro-angiogenic agent, exemplary integrin binding sequence can be a RGD motif, a RGD related motif or a non-RGD integrin recognition motif.

In another aspect, the invention is directed to an isolated nucleic acid, which nucleic acid encodes a protein or peptide selected from the group consisting of the entire extracellular domain of the NCAM L1 or a functional derivative or fragment thereof that substantially retains its binding affinity with the integrin, a NCAM L1 comprising the Ig-like domains 4-6 (Ig 4-6) or a functional derivative or fragment thereof that substantially retains its binding affinity with the integrin, a protein or a peptide that specifically binds to an antibody that is raised against a peptide having the following amino acid sequence: PSITWRGDGRDLQEL and a peptide having the following amino acid sequence: PSITWRGDGRDLQEL.

In still another aspect, the invention is directed to a combination, which combination comprises: a) an effective amount of an integrin binding pro-angiogenic agent; and b) an effective amount of another angiogenic molecule.

In yet another aspect, the invention is directed to a method for enhancing angiogenesis, which method comprises administering an effective amount of a combination comprising: a) an effective amount of an integrin binding pro-angiogenic agent; and b) an effective amount of another angiogenic molecule, to a mammal, wherein angiogenesis is desirable, thereby enhancing angiogenesis in said mammal.

In yet another aspect, the invention is directed to a method for enhancing angiogenesis, which method comprises administering an effective amount of an integrin antagonist to a mammal, wherein angiogenesis is desirable, thereby enhancing angiogenesis in said mammal. Preferably, the integrin antagonist is an integrin anti-sense oligonucleotide, an anti-integrin antibody, a soluble integrin, or a derivative or fragment thereof, or an agent that reduces or inhibits production of the integrin.

In yet another aspect, the invention is directed to a combination, which combination comprises: a) an effective amount of an integrin antagonist; and b) an effective amount of another angiogenic molecule.

### BRIEF DESCRIPTION OF THE DRAWINGS

FIG. 1 depicts induction of angiogenesis by soluble L1-fusion protein containing the fourth, fifth, and sixth Ig-like domains of L1 (Ig 4-6). a. GST fusion proteins were made covering the entire extra-cellular domain of L1 and consisted of immunoglobulin like domains 1 through 3 (Ig1 3), immunoglobulin like domains 4 through 6 (Ig4-6), and FN type III like domains 1-5 (FN1 5). b. The L1-fusion proteins (Ig 1-3, Ig 4-6 or FN1-5) were tested for their ability to induce an angiogenic response in the chick chorioallantoic membrane (CAM) model. Filter discs each received 0.5 mg of Ig4-6 fusion protein (10 ml/disc) or equimolar amounts of the Ig 1-3, or FN1-5 fusion proteins. Negative or positive control groups received filter discs saturated with media alone or with bFGF respectively. Angiogenesis was quantified by counting the number of vessel branch points within the area of membrane immediately beneath the disc. The number of vessel branch points counted in the control group receiving media alone has been subtracted. Treatment groups consisted of a minimum of six embryos and experiments were performed on three separate occasions. c. Photographs of chorioallantoic membrane demonstrating the induction of angiogenesis by the L1 Ig 4-6 fusion protein. Results obtained using bFGF or media alone are shown for comparison. Note that fine and highly branched angiogenic vessels can be distinguished from large preexisting vessels. Dotted lines delineate the area of the filter disc placed on the membrane. Excised membrane beneath and adjacent to the filter discs were photographed using an Olympus SZH10 Research Stereo microscope at 15×.

FIG. 2 depicts induction of angiogenesis by a L1-RGD peptide and by a function blocking antibody to β1 integrins. a. The L1 peptide PSITWRGDGRDLQEL or the mutant L1 peptide PSITWRADGRDLQEL were tested for their ability to induce an angiogenic response in the chick CAM model. b. Function blocking antibodies to chick avβ3 (LM609) or to chick β1-intgrins (CSAT) were also tested. Filter discs were saturated with 10 ml of media alone or 10 ml media containing different amounts of the L1-peptides (0.01-5 mg/disc) or the anti-integrin antibodies (1 10 mg/disc). Angiogenesis was quantified by counting the number of vessel branch points within the area of membrane immediately beneath the disc. The number of vessel branch points counted in the control group receiving media alone has been subtracted. c. Photographs of chorioallantoic membrane demonstrating the induction of angiogenesis by the L1-RGD peptide and CSAT. Results obtained using LM609 are shown for comparison. Dotted lines delineate the area of the filter disc placed on the membrane. Excised membrane beneath and adjacent to the filter discs were photographed using an Olympus SZH10 Research Stereo microscope at 15×.

FIG. 3 depicts induction of angiogenesis by L1 cleavage products. a. A purified glycosylated L1-His fusion protein consisting of the entire extracellular domain of L1 was incubated with immobilized trypsin for 15-60 minutes. Untreated and digested L1 (trypsin free) was then analyzed by SDS polyacrylamide gel electrophoresis (5-15% gradient). b. Sites of trypsin cleavage were determined by amino terminal sequencing and are shown schematically. c. Cleaved L1-fragments were tested for their ability to induce an angiogenic response in the CAM model. Negative or positive control groups received filter discs saturated with trypsin-treated media alone or with bFGF respectively. Angiogenesis was quantified by counting the number of vessel branch points within the area of membrane immediately beneath the disc.

FIG. 4 depicts melanoma and neuroblastoma cells shed significant amounts of soluble L1. a. The amount of soluble L1 shed into serum-free tumor conditioned media over a 72 hour period by M21 melanoma cells or SK-N-AS neuroblastoma cells was quantified using a two antibody sandwich immunoassay with anti-L1 monoclonal antibody 5G3 and an affinity purified anti-L1 polyclonal antibody. b. Levels of L1 in the serum of normal individuals (n=8) or patients with neuroblastoma (stage I-IV, n=29) was determined also by two antibody sandwich immunoassay with anti-L1 monoclonal antibody 5G3 and an affinity purified anti-L1 polyclonal antibody. Absolute amounts of L1 were determined by reference to a standard curve obtained using purified L1 consisting of the entire extracellular domain of L1 (not shown).

### MODES OF CARRYING OUT THE INVENTION

A. Definitions

Unless defined otherwise, all technical and scientific terms used herein have the same meaning as is commonly understood by one of ordinary skill in the art to which this invention belongs. All patents, applications, published applications and other publications and sequences from GenBank and other databases referred to herein are incorporated by reference in their entirety.

As used herein, “integrins” refers to a family of cell membrane glycoproteins that are heterodimers composed of α- and β-chain subunits. They serve as glycoprotein receptors involved in cell-cell or cell-substrate adhesion, e.g., the mediation of adhesion of neutrophils to endothelial cells, or to extracellular matrix such as collagen.

As used herein, an “integrin binding pro-angiogenic agent” refers to a substance that exerts its angiogenic effect via its binding with an integrin, or an integrin containing complex.

As used herein, an “antagonist of integrin (or integrin antagonist)” refers to a substance that decreases production and/or anti-angiogenic function of integrin. Such an antagonist can decrease production of integrin by decreasing transcription and or translation of an integrin gene, or by decreasing post-translational modification and/or cellular trafficking of an integrin precursor, or by shortening half-life of an integrin protein. Such an antagonist can also decrease anti-angiogenic function of integrin by decreasing potency of integrin's anti-angiogenic activity, or by decreasing sensitivity of an integrin's natural ligand in an angiogenesis pathway, or by increasing potency of an integrin's antagonist.

As used herein, “antisense polynucleotides” refer to synthetic sequences of nucleotide bases complementary to mRNA or the sense strand of double stranded DNA. Admixture of sense and antisense polynucleotides under appropriate conditions leads to the binding of the two molecules, or hybridization. When these polynucleotides bind to (hybridize with) mRNA, inhibition of protein synthesis (translation) occurs. When these polynucleotides bind to double stranded DNA, inhibition of RNA synthesis (transcription) occurs. The resulting inhibition of translation and/or transcription leads to an inhibition of the synthesis of the protein encoded by the sense strand.

As used herein, an “integrin antisense oligonucleotide” refers to any oligomer that prevents production or expression of integrin polypeptide. The size of such an oligomer can be any length that is effective for this purpose. In general, the antisense oligomer is prepared in accordance with the nucleotide sequence of a portion of the transcript of integrin that includes the translation initiation codon and contains a sufficient number of complementary nucleotides to block translation.

As used herein, a “soluble integrin” refers to any fragment of an integrin protein that is not membrane-bound, e.g., due to the lack of the transmembrane domain, but nevertheless substantially retains its binding affinity with natural homo- or hetero-binding ligand. Preferably, the soluble integrin also lacks any intracellular domain of an integrin that is involved in its signal transduction function.

As used herein, “antibody” includes polyclonal or monoclonal antibodies, single-chain antibodies and other antibody fragments, such as Fab fragments, which are composed of a light chain and the variable region of a heavy chain.

As used herein, “humanized antibodies” refer to antibodies that are modified to include “human” sequences of amino acids so that administration to a human will not provoke an immune response. Methods for preparation of such antibodies are known. For example, the hybridoma that expresses the monoclonal antibody is altered by recombinant DNA techniques to express an antibody in which the amino acid composition of the non-variable regions is based on human antibodies. Computer programs have been designed to identify such regions.

As used herein, “production by recombinant means” refers to production methods that use recombinant nucleic acid methods that rely on well known methods of molecular biology for expressing proteins encoded by cloned nucleic acids.

As used herein, “neural cell adhesion molecule L1 (NCAM L1)” refers to a neural cell adhesion molecule that belongs to the IgSF superfamily. It is intended that NCAM L1 includes those variants with conservative amino acid substitutions that do not substantially alter its integrin binding activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Bejacmin/Cummings Pub. co., p.224).

As used herein, a “functional derivative or fragment of NCAM L1 that substantially retains its binding affinity with the integrin” refers to a derivative or fragment of NCAM L1 that still substantially retains its integrin-binding and pro-angiogenic activity. Normally, the derivative or fragment retains at least 1%, 10%, 20%, 30%, 40%, 50% of its binding affinity with the integrin. Preferably, the derivative or fragment retains at least 60%, 70%, 80%, 90%, 95%, 99% and 100% of its binding affinity with the integrin.

As used herein, a “soluble NCAM L1” refers to any fragment of a NCAM L1 protein that is not membrane-bound, e.g., due to the lack of the transmembrane domain, but nevertheless substantially retains its binding affinity with integrin. Preferably, the soluble NCAM L1 also lacks any intracellular domain of a NCAM L1 that is involved in its signal transduction function.

As used herein, “an effective amount of an integrin binding pro-angiogenic agent” refers to an amount of the agent that is angiogenic but not so high as to become anti-angiogenic. Such amount should be determined in view of the agent used, the target integrin and the route of administration. If necessary, the amount can be empirically determined, e.g., by various in vitro, in vivo or clinical models of angiogenesis assays known in the art. If the agent is used to treat a disease or disorder by enhancing angiogenesis, the amount refers to an amount sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

As used herein, “an effective amount of an antagonist of an integrin” refers to an amount of the antagonist that is angiogenic but not so high as to become anti-angiogenic. Such amount should be determined in view of the antagonist used, the target integrin and the route of administration. If necessary, the amount can be empirically determined, e.g., by various in vitro, in vivo or clinical models of angiogenesis assays known in the art. If the integrin antagonist is used to treat a disease or disorder by enhancing angiogenesis, the amount refers to an amount sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

As used herein: stringency of hybridization in determining percentage mismatch is as follows:

1) high stringency: 0.1×SSPE, 0.1% SDS, 65° C.

2) medium stringency: 0.2×SSPE, 0.1% SDS, 50° C.

3) low stringency: 1.0×SSPE, 0.1% SDS, 50° C.

It is understood that equivalent stringencies may be achieved using alternative buffers, salts and temperatures.

As used herein, “an ischemic disease” refers to a disease or disorder characterized by a decrease in the blood supply to a body organ, tissue, or part caused by constriction or obstruction of the blood vessels.

As used herein, a “combination” refers to any association between two or among more items.

As used herein, a “composition” refers to any mixture of two or more products or compounds. It may be a solution, a suspension, liquid, powder, a paste, aqueous, non-aqueous or any combination thereof.

For clarity of disclosure, and not by way of limitation, the detailed description of the invention is divided into the subsections that follow.

B. Methods for Enhancing Angiogenesis

In one aspect, the invention is directed to a method for enhancing angiogenesis, which method comprises administering an effective amount of an integrin binding pro-angiogenic agent or an antagonist of an integrin to a mammal, wherein angiogenesis is desirable, thereby enhancing angiogenesis in said mammal.

In one specific embodiment, the integrin binding pro-angiogenic agent is a protein, a polypeptide or a peptide. Preferably, the protein, polypeptide or peptide pro-angiogenic agent binds to an integrin containing the β1 subunit. Also preferbaly, the protein, polypeptide or peptide pro-angiogenic agent contains an integrin binding sequence. More preferbaly, the integrin binding sequence contains the RGD motif, a RGD related motif or a non-RGD integrin recognition motif. Exemplary RGD motifs include PSITWRGDGRDLQEL. Exemplary non-RGD integrin recognition motifs include RRETAWA (Koivunen et al., J. Cell. Biol., 124:373-380 (1994)) and GSQRKHSKR and QVKGHLR (Silletti et al., J. Cell Biology, 149:11485-1501 (2000)).

In another specific embodiment, the integrin binding pro-angiogenic agent is a small molecule agent.

Any integrin can be used as the target to enhance desired angiogenesis. For example, the integrin comprising a β1, β2, β3, β4, β5, β6, β7 or β8 subunit can be used as the target. Preferably, the integrin comprising a β1 subunit can be used as the target. Also preferably, the integrin comprising α5β1 or αvβ1 subunits can be used as the target. In a specific embodiment, the integrin comprising the following β subunits can be used as the target: AF224337 (Ictalurus punctatus beta-1 integrin mRNA); AF060203 (Biomphalaria glabrata beta integrin); AF115376 (Mus musculus integrin beta-6); AF022110 (Mus musculus integrin beta-5); RNU60096 (Rattus norvegicus sciatic nerve integrin beta 4); L13305 (Drosophila melanogaster integrin beta); AF078802 (Strongylocentrotus purpuratus integrin beta L); FCU27351 (Felis catus beta-1 integrin); AF059607 (Lytechinus variegatus beta-C); SPU77584 (Strongylocentrotus purpuratus integrin beta G); RNU12309 (Rattus norvegicus integrin beta-1); CEU19744 (Caenorhabditis elegans integrin beta pat-3); M62880 (Human integrin beta-7); M68892 (Human integrin beta-7 subunit); J05633 (Human integrin beta-5); M35011 (Human integrin beta-5); M20180 or J03736 (X. laevis integrin beta-1); M20140 or J03736 (X. laevis integrin beta-1); M95633 (Mouse integrin beta-7); M95632 (Mouse integrin beta-7); M73780 (Human integrin beta-8); M73781 (Oryctolagus cuniculus integrin beta-8); L13591 (Xenopus laevis integrin beta-3); and M68903 (Mouse integrin beta-7).

Any integrin antagonist can be used in the present method. For example, the integrin antagonist can be an integrin anti-sense oligonucleotide, an anti-integrin antibody, a soluble integrin, or a derivative or fragment thereof, or an agent that reduces or inhibits production and/or anti-angiogenic function of the integrin. In a specific embodiment, integrin anti-sense oligonucleotides, anti-integrin antibodies, and soluble integrins, or derivatives or fragments thereof, that are derived from or raised against the above-described integrin nucleic acids or encoded proteins can be used.

The anti-integrin antibody can be a polyclonal or a monoclonal antibody. Preferably, the anti-integrin antibody is CSAT, AG89 (Takagi et al., J. Biochem. (Tokyo), 121(5):914-21 (1997), QE.2E5 (Faull et al., J. Biol. Chem., 271(41):25099-106 (1996)), mAb 13 (Mould et al., J. Biol. Chem., 271(34):20365-74 (1996)), or NaM160-1A3 (Richard et al., Xenotransplantation, 5(1):75-83 (1998)).

Any integrin binding pro-angiogenic agent can be used in the present method. For example, an agent that perturbs integrin ligation resulting in a lessening of vascular cell adhesivity can be used. In another example, an agent that induces a conformational change in the integrin and exposes otherwise cryptic ligand-induced binding sites (LIBS), e.g. a cryptic collagen type I binding site, can be used. In still another example, an agent that redistributes the integrin into focal contacts can be used. In yet another example, the pro-angiogenic agent promotes vascular cell migration and/or protease activity can be used.

In a specific embodiment, the integrin binding pro-angiogenic agent is a neural cell adhesion molecule L1 (NCAM L1) or a functional derivative or fragment thereof that substantially retains its binding affinity with the integrin, or a nucleic acid encoding said NCAM L1 or functional derivative or fragment thereof.

Any NCAM L1, or a functional derivative or fragment thereof, that can function as an integrin binding pro-angiogenic agent, and any nucleic acids encoding such NCAM L1, or functional derivative or fragment thereof, can be used in the present methods.

For example, NCAM L1 proteins with the following GenBank accession numbers can be used: T30532 (Fugu rubripes); T30581 (zebra fish); S36126 (rat); A43425 (chicken); S05479 (mouse); A41060 (human); NP_{—}032504 (Mus musculus); NP_{—}006605 (close homologue of L1 sapiens); NP_{—}000416 (Homo sapiens); AAF22153 (Mus musculus); CAB57301 (Mus musculus); P32004 (HUMAN); Q05695 (RAT); P11627 (MOUSE); AAD28610 (Cercopithecus aethiops); CAB37831 (Homo sapiens); AAC51746 (Homo sapiens); AAC15580 (Fugu rubripes); AAC14352 (Homo sapiens); CAA96469 (Fugu rubripes); CAA82564 (Homo sapiens); CAA41576 (Homo sapiens); 1411301A; CAA42508 (Homo sapiens); CAA41860 (Rattus norvegicus); AAA99159 (Carassius auratus); CAA61491 (Danio rerio); CAA61490 (Danio rerio); AAA59476 (Homo sapiens); AAA36353 (Homo sapiens). In addition, any proteins derived from, or are portion of, the above NCAM L1 proteins that still substantially retain their integrin antagonizing and/or binding activities can be used. Preferably, such NCAM L1 derivatives or fragments can be recognized by antibodies that specifically recognize the NCAM L1 proteins from which the derivatives or fragments originate.

Similarly, nucleic acids encoding NCAM L1 proteins with the following GenBank accession numbers can be used: AC005775 (Homo sapiens); AC 004690 (Homo sapiens); M28231 (Drosophila melanogaster neuroglian precursor); AH006326 (Drosophila melanogaster neuroglian (nrg), alternative splice products); AF050085 (Drosophila melanogaster neuroglian (nrg) gene; AF172277 (Homo sapiens); AF133093 (Mus musculus); AJ239325 (Homo sapiens); AL021940 (Homo sapiens); AF129167 (Chlorocebus aethiops); AJ011930 (Homo sapiens); U52112 (Homo sapiens); M97161 (Rattus norvegicus); AC005626 (Homo sapiens); AF026198 (Fugu rubripes); M77640 (Homo sapiens); U55211 (Carassius auratus); M74387 (Human). In addition, any nucleic acids derived from, or are portion of, the above nucleic acids encoding NCAM L1 that still substantially retain their integrin antagonizing and/or binding activities can be used. Preferably, such NCAM L1 nucleic acid derivatives or fragments can hybridize under low, middle or high stringency with the NCAM L1 nucleic acids from which the derivatives or fragments originate.

Preferably, the NCAM L1 is a soluble NCAM L1 or a functional derivative or fragment thereof that substantially retains its binding affinity with the integrin. Also preferably, the NCAM L1 comprises the Ig-like domains 4-6 (Ig 4-6) of the extracellular domain of the NCAM L1 or a functional derivative or fragment thereof that substantially retains its binding affinity with the integrin.

In another specific embodiment, the integrin binding pro-angiogenic agent is a protein or a peptide that specifically binds to an antibody that is raised against a peptide having the following amino acid sequence: PSITWRGDGRDLQEL. Preferably, the peptide has the following amino acid sequence: PSITWRGDGRDLQEL. Antibodies, whether polyclonal or monoclonal antibodies, can be raised against the desired peptides by any methods known in the art (see e.g., Antibody Production: Essential Techniques, Delves, Wiley, John & Sons, Inc., 1997; Basic Methods in Antibody Production and Characterization, Howard and Bethell, CRC Press, Inc., 1999; and Monoclonal Antibody Production Techniques and Applications: Hybridoma Techniques, Schook, Marcel Dekker, 1987).

The present methods can be used to treat, either prophylactically or therapeutically, mammals with diseases or disorders associated with deficient angiogenesis. Examples of such diseases or disorders include, but are not limited to, ischemic disease or wound healing disorders.

Any mammals, such as, mice, rats, rabbits, cats, dogs, pigs, cows, ox, sheep, goats, horses, monkeys and other non-human primates, with ischemic disease or wound healing disorders, can be treated with the present methods. Preferably, humans with ischemic disease or wound healing disorders are treated with the present methods.

C. Compositions, Combinations and Combinatorial Methods for Enhancing Angiogenesis

In another aspect, the invention is directed to an isolated protein or peptide, which protein or peptide is the entire extracellular domain of the NCAM L1 or a functional derivative or fragment thereof that substantially retains its binding affinity with the integrin, a NCAM L1 comprising the Ig-like domains 1-3 (Ig 1-3), Ig-like domains 4-6 (Ig 4-6) or Ig-like domains 1-6 (Ig 1-6) of the extracellular domain of the NCAM L1 or a functional derivative or fragment thereof that substantially retains its binding affinity with the integrin, a protein or a peptide that specifically binds to an antibody that is raised against a peptide having the following amino acid sequence: PSITWRGDGRDLQEL and a peptide having the following amino acid sequence: PSITVRGDGRDLQEL. Preferably, such isolated protein or peptide is formulated as a pharmaceutical composition with a pharmaceutically acceptable carrier or excipient.

In still another aspect, the invention is directed to an isolated nucleic acid, which nucleic acid encodes a protein or peptide is the entire extracellular domain of the NCAM L1 or a functional derivative or fragment thereof that substantially retains its binding affinity with the integrin, a NCAM L1 comprising the Ig-like domains 1-3 (Ig 1-3), Ig-like domains 4-6 (Ig 4-6) or Ig-like domains 1-6 (Ig 1-6) of the extracellular domain of the NCAM L1 or a functional derivative or fragment thereof that substantially retains its binding affinity with the integrin, a protein or a peptide that specifically binds to an antibody that is raised against a peptide having the following amino acid sequence: PSITWRGDGRDLQEL and a peptide having the following amino acid sequence: PSITWRGDGRDLQEL. Preferably, such isolated nucleic acid is formulated as a pharmaceutical composition with a pharmaceutically acceptable carrier or excipient.

In yet another aspect, the invention is directed to a combination, which combination comprises: a) an effective amount of an integrin binding pro-angiogenic agent or an integrin antagonist; and b) an effective amount of another angiogenic molecule. Preferably, such combination is formulated as a pharmaceutical composition with a pharmaceutically acceptable carrier or excipient.

Any integrin binding pro-angiogenic agent or integrin antagonist, including the ones described in the above Section B, can be used in the combination. Any angiogenic molecule, other than an integrin antagonist, can be used a second component of the combination. For example, the other angiogenic molecule can be an angiogenic cytokine, or a functional derivative or fragment thereof, that substantially retains its angiogenic activity, or a nucleic acid encoding an angiogenic cytokine, or a functional derivative or fragment thereof, that substantially retains its angiogenic activity. Preferably, the angiogenic cytokine is an acidic fibroblast growth factor (aFGF), an angiopoietin, a basic fibroblast growth factor (bFGF), a heparin-binding epidermal growth factor (HB-EGF), an insulin-like growth factor (IGF), a placental growth factor (PIGF), a platelet derived growth factor (PDGF), a scatter factor hepatocyte growth factor (HGF), a transforming growth factor-beta (TGF-beta) and a vascular endothelial growth factor (VEGF), or a functional derivative or fragment thereof that substantially retains its angiogenic activity, or a nucleic acid encoding an angiogenic cytokine, or a functional derivative or fragment thereof that substantially retains its angiogenic activity.

In yet another aspect, the invention is directed to a method for enhancing angiogenesis, which method comprises administering an effective amount of the above-described combination to a mammal, wherein angiogenesis is desirable, thereby enhancing angiogenesis in said mammal.

The formulation, dosage and route of administration of the above-described compositions, combinations, preferably in the form of pharmaceutical compositions, can be determined according to the methods known in the art (see e.g. Remington: The Science and Practice of Pharmacy, Alfonso R. Gennaro (Editor) Mack Publishing Company, April 1997; Therapeutic Peptides and Proteins: Formulation, Processing, and Delivery Systems, Banga, 1999; and Pharmaceutical Formulation Development of Peptides and Proteins, Hovgaard and Frkjr (Ed.), Taylor & Francis, Inc., 2000; Medical Applications of Liposomes, Lasic and Papahadjopoulos (Ed.), Elsevier Science, 1998; Textbook of Gene Therapy, Jain, Hogrefe & Huber Publishers, 1998; Adenoviruses: Basic Biology to Gene Therapy, Vol. 15, Seth, Landes Bioscience, 1999; Biopharmaceutical Drug Design and Development, Wu-Pong and Rojanasakul (Ed.), Humana Press, 1999; Therapeutic Angiogenesis: From Basic Science to the Clinic, Vol. 28, Dole et al. (Ed.), Springer-Verlag New York, 1999). The compositions, combinations or pharmaceutical compositions can be formulated for oral, rectal, topical, inhalational, buccal (e.g., sublingual), parenteral (e.g., subcutaneous, intramuscular, intradermal, or intravenous), transdermal administration or any other suitable route of administration. The most suitable route in any given case will depend on the nature and severity of the condition being treated and on the nature of the particular composition, combination or pharmaceutical composition which is being used. For instance, in treating ischemic diseases, arterial gene transfer can be used (Isner and Asahara, Frontiers in Bioscience, 3:e49-69 (1998)).

The efficacy and/or toxicity of the above-described compositions, combinations or pharmaceutical compositions can also be assessed by the methods known in the art (See generally, O'Reilly, Investigational New Drugs, 15:5-13 (1997)). For example, the in vitro angiogenesis assays based on target compound's ability to inhibit endothelial cell proliferation, migration, and tube formation in vitro can be used. Alternatively, the in vivo angiogenesis assays such as the chicken chorioallantoic membrane (CAM) assay and the disc angiogenesis assays can be used. Preferably, the established pre-clinical models for the evaluation of angiogenesis inhibitors in vivo such as corneal angiogenesis assays, primate model of ocular angiogenesis, metastasis models, primary tumor growth model and transgenic mouse model of tumor growth can be used.

The following example is included for illustrative purposes only and is not intended to limit the scope of the invention.

D. Examples

Neovascularization or angiogenesis is a quintessential component of many normal or pathogenic processes from development and wound repair to inflammation and tumorigenesis. The development of a new blood vessel is a complex multistage process involving different temporally regulated elements from proteolysis, migration, proliferation, to lumen formation and differentiation. Almost every element of this cascade is profoundly regulated by the extracellular matrix that constitutes the microenvironment of the vascular cell.

As primary mediators of the informational cues provided by the ECM, vascular integrins are of primary importance. Indeed integrins have been implicated in every aspect of the angiogenic process from regulators of proteolysis, and vascular cell proliferation and survival, to mediators of motility and tubulogenesis¹⁻⁸. Given the central role of integrins, it is intuitive that antagonists of integrin function will disrupt blood vessel development. Indeed antibodies to β1-integrins and to the vitronectin receptor αvβ3, have been shown to disrupt vasculogenesis and angiogenesis respectively^{9,10}. However, it is also clear from a number of in vitro studies that integrin antagonists may also potentiate elements of the angiogenic process including proteolysis^{1,11}, and tubulogenesis¹².

While the significance of the integrin superfamily in angiogenesis is well established the role of the immunoglobulin superfamily (IgSF) is less defined. Indeed, while a plethora of IgSF members have been described on vascular cells, the functional significance of these cell adhesion molecules (CAMs) has mainly been linked to the potentiation of extravasation. However, as mediators of cell-cell adhesion one might predict a role for IgSF members in the inter-cellular interactions required for lumen or vessel formation and maintenance. In this regard, recent findings do support a role for PECAM-1 (CD31) in homotypic endothelial cell interactions required for lumen formation13. Of immediate relevance to this study is the recent finding that a soluble IgSF member, VCAM-1, can in fact induce an angiogenic response and that this proangiogenic activity is likely to be linked to the ligation of vascular α4β1^{14,15}.

The observation that soluble VCAM-1 can induce neovascularization via its interaction with a vascular integrin, presents some interesting questions, including whether this is an unique property of this CAM or if other IgSF members are also proangiogenic and other vascular integrins can serve as target ligands for the induction of such a response. In this regard, we and others have recently documented that the neural CAM L1 is another example of an IgSF member that can interact with integrins¹⁶⁻¹⁸.

L1 is a multidomain glycoprotein consisting of six immunoglobulin-like (Ig-like) domains followed by five fibronectin type III-like (FN-like) repeats^{19,20}. A single transmembrane region links this extracellular domain to a short highly conserved cytoplasmic tail¹⁹. Significantly, human L1 contains a single RGD integrin recognition motif within the sixth Ig-like domain²¹ and several putative protease cleavage sites that facilitate posttranslational cleavage^{22,23}. L1 is described as a neural cell adhesion molecule based on its association with cells of neural origin, including post mitotic neurons of the central and peripheral nervous systems^{24,25}. However, L1 has also been described on a wide range of tumors of different histological origin^{26,28} and on cells of the lymphoid or myelomonocytic series²⁹.

Based on the findings that L1 can interact with vascular integrins¹⁸, and is expressed and shed by many neuroectodermal tumors²⁶, we elected to determine whether L1 is an example of an IgSF member that can induce an angiogenic response. Utilizing the chick chorioallantoic membrane (CAM) model we demonstrate that a defined soluble fragment of L1 can induce a significant angiogenic response. This proangiogenic fragment contains a RGD motif that is recognized by the integrins αvβ3 and αvβ1¹⁸. Significantly, we are able to confirm the importance of the RGD motif by demonstrating that a short L1-peptide containing this motif also induces an angiogenic response. Since the RGD peptide alone is sufficient for the induction of angiogenic response we propose a novel mechanism for the induction of angiogenesis based on the perturbation of integrin binding. Adding support to this concept we also show that a function-blocking antibody to the b1-integrin subunit can also promote angiogenesis.

Results

Induction of Angiogenesis by a Soluble L1-Fragment

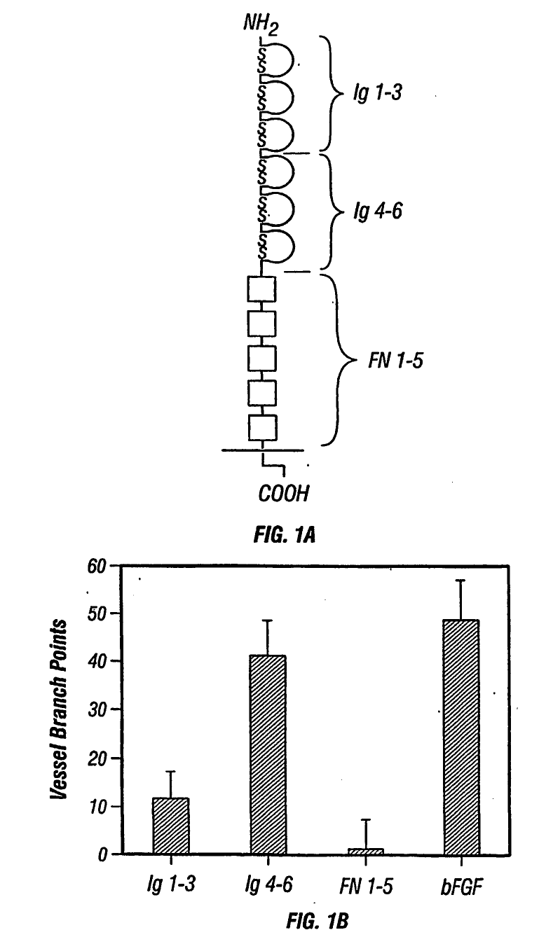

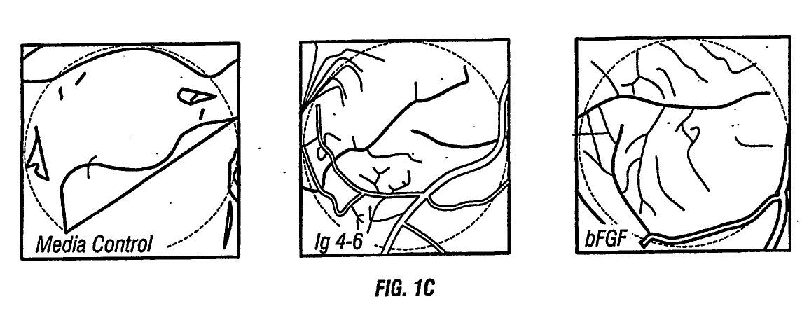

In order to determine whether soluble L1 polypeptides can induce angiogenesis, we tested three L1 GST fusion proteins that together span the entire extracellular domain of L1. These fusion proteins consist of Ig-like domains 1-3 (Ig 1-3), Ig-like domains 4-6 (Ig 4-6) and all the fibronectin like domains (FN 1-5) (FIG. 1a). The generation and characterization of these fusion proteins has been described in detail elsewhere³⁰. The ability of these fusion proteins to induce angiogenesis was assessed in the chick chorioallantoic model as indicated in the methods section.

Most notable is the induction of a significant angiogenic response by the fragment containing Ig-like domains 4 through 6 (Ig 4-6) (FIGS. 1b & c). Such a response was not observed with equimolar amounts of the fibronectin-like domains of L1 (FN 1-5), and immunoglobulin-like domains 1 through 3 (Ig 1-3) induced only a limited response (FIG. 1b). The response induced by Ig 4-6 was comparable to that induced by bFGF used at a concentration optimal for the induction of an angiogenic response (FIGS. 1b & c). A significant angiogenic response to Ig 4-6, but not to Ig 1-3 or FN 1-5, argues against a role for GST or any trace amounts of endotoxin in the fusion protein preparations. In this regard, all of the fusion proteins were generated under similar experimental conditions and contained comparable trace levels of endotoxin when applied to the CAM (FN1-5=0.54 EU/ml; Ig1-3=0.12 EU/ml; Ig4-6=0.152 EU/ml).

A Soluble 15-mer L1-RGD Peptide is Sufficient for the Induction of Angiogenesis

An important property of the proangiogenic L1-fusion protein Ig4-6 is the presence of a single RGD integrin recognition motif. We have previously shown that L1-fragments containing this motif (Ig 4-6 and Ig 6 alone) can support significant integrin-dependent adhesion and migration^{16,18}. Furthermore, we have shown that endothelial cells can recognize this RGD motif using αvβ3 or αvβ1^{16,18}. In order to assess the importance of the L1 RGD motif, we synthesized a 15 mer RGD based on the L1 sequence PSITWRGDGRDLQEL.

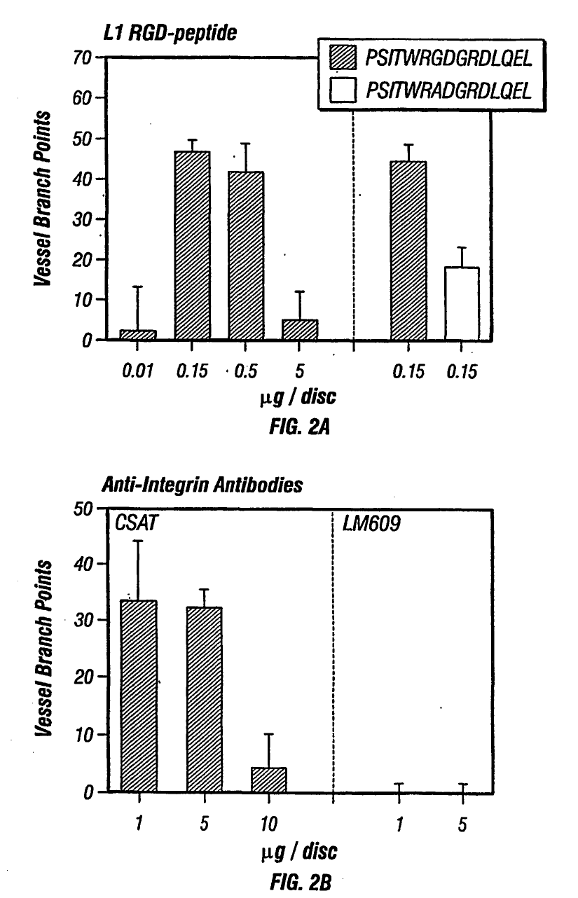

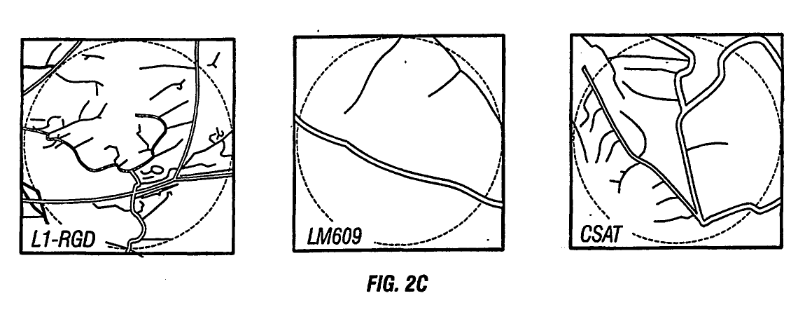

Remarkably this L1-RGD peptide was also found to be proangiogenic within a defined concentration range (FIGS. 2a & c). It is important to note that this L1-RGD peptide was not effective at a high concentration (FIG. 2a). An additional 15-mer L1 peptide containing the mutation RGD to RAD (i.e., PSITWRADGRDLQEL) was significantly less proangiogenic when compared with an equivalent concentration of the wildtype peptide (FIG. 2a). Interestingly, this mutant peptide still displayed some angiogenic activity but was also found to have retained some integrin-binding activity (data not shown). It should be noted that the blood vessels formed in response to the L1 RGD peptide were generally finely structured, being less obvious or developed, than those formed in response to the bFGF (FIG. 2c).

The observation that a soluble L1-RGD peptide can induce angiogenesis suggests a mechanism that involves perturbation of integrin function. If disruption of integrin ligation is responsible then one should also be able to induce angiogenesis with function blocking anti integrin antibodies. Since we have identified both αvβ3 and αvβ1 as potential target ligands we tested two function-blocking antibodies reactive with chick αvβ3 (LM609) or chick β1-integrins (CSAT). No antibody specific for chick αvβ1 is currently available. Significantly, while we observed no response with the avβ3-specific antibody, the anti-β1 antibody did indeed induce a significant angiogenic response (FIGS. 2b & c). Importantly, this anti-β1 antibody gave a dose response profile similar to that of the L1-RGD peptide with high concentrations of the antibody being ineffective or inhibitory (FIG. 2b). These data suggest a mechanism involving an interaction between soluble L1-RGD peptide or L1-RGD fragment and a β1-integrin. In this regard, we have observed that both the proangiogenic L1-Ig4-6 fragment and the L1-RGD peptide are recognized by αvβ1 expressed by endothelial cells¹⁸. These observations suggest a novel mechanism for the induction of angiogenesis with broad implications for the potentiation of angiogenesis by other integrin-reactive polypeptides including those derived from proteolysed extracellular matrix.

Induction of Angiogenesis by L1 Cleavage Products

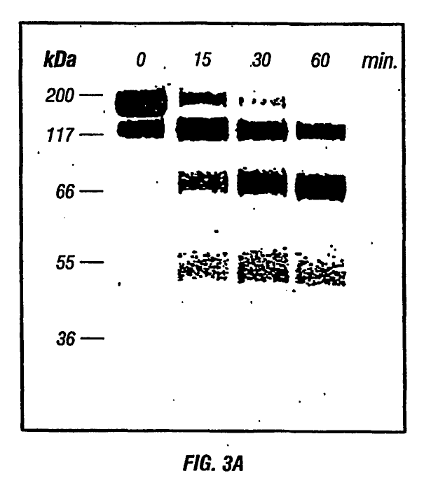

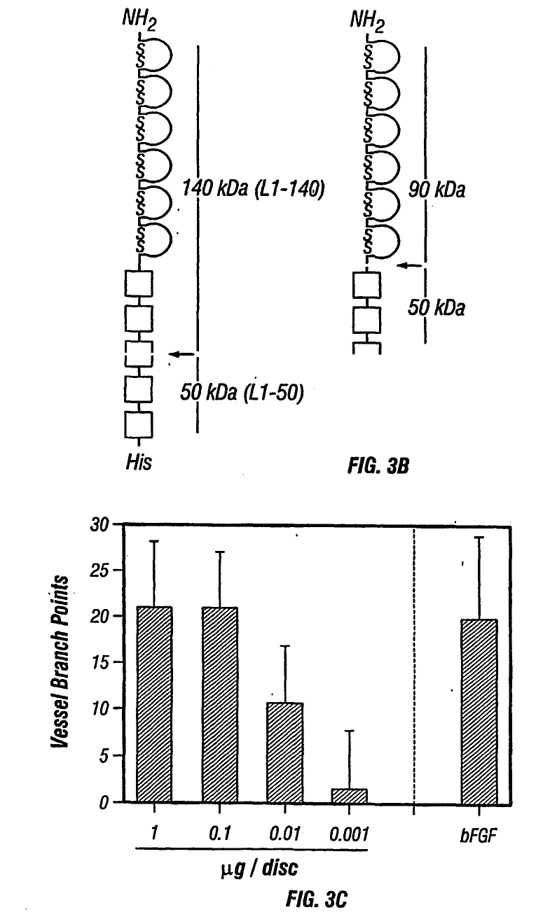

Soluble L1 cleavage products have been described in vivo^{31,32} and are likely to be the result of posttranslational proteolysis^{22,23}. In vitro studies have shown that trypsin will generate L1 fragments that are equivalent in size and molecular origin as those found in vivo^{22,23}. Based on these findings we determined whether a L1-construct consisting of the entire extracellular domain of L1 (L1-ECD-His) could induce angiogenesis after mild trypsinization. The L1-ECD-His fusion protein was produced in eukaryotic cells as a glycosylated protein and was purified from cell culture media as full length product of 190 kDa and as a lesser L1 cleavage product of 140 kDa (L1 140) (FIG. 3a). After mild trypsinization the L1-ECD-His fusion protein was broken down into a series of large fragments (L1-140, L1-95, & L1-50) (FIG. 3a). Amino-terminal sequencing confirmed that these products are the result of cleavage at a site in the middle of the third FN-like domain (L1-140, L1-50) and at a site between Ig-like domain 6 and FN-like domain-1 (L1-95) (FIG. 3b). Importantly, cleavage products equivalent to L1-140 and L1-50 have been described in vivo³¹⁻³³. L1-cleavage products resulting from a 30 minute digestion (FIG. 3a) were tested in the CAM model and in accordance with our observation with the L1 Ig-4-6 fragment were found to be proangiogenic (FIG. 3c). Optimal activity was detected in the range of 1-0.1 mg/disc (FIG. 3c).

Neuroectodermal Tumors are an Important Source of Soluble L1 Fragments

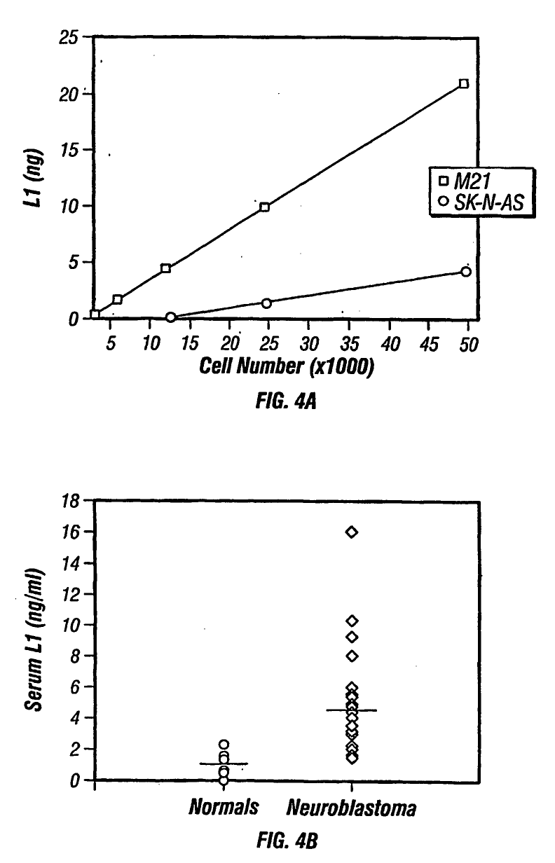

The physiological context of L1-release is important for understanding its potential role in the induction of angiogenesis. Previously it has been reported that many neuroectodermal tumors express L1 including a variety of melanoma and neuroblastoma cell lines²⁷. To determine whether such lines shed L1 and to quantify the amounts of soluble L1 produced we established a two antibody ELISA using both a monoclonal (5G3) and an affinity purified anti-L1 polyclonal antibody. The production of soluble L1 by both a melanoma (M21) and a neuroblastoma cell line (SK-N-AS) was assessed after 72 hours in serum free media (FIG. 4a). 5×103 M21 or SK-N-AS cells produced approximately 20 and 5 ng of soluble L1, respectively (FIG. 4a). Since the application of 1-0.1 mg of cleaved-L1 (FIG. 3c) was sufficient to induce a response in the chick chorioallantoic membrane, the L1-production of a small tumor (>5×106 cells) should theoretically be sufficient to promote an angiogenic response.

To further establish pathophysiological relevance we determined whether levels of serum L1 are elevated in patients with neuroblastoma (stage I through IV). Consistent with significant local production of soluble L1 we detected significantly elevated serum L1 levels in the majority of these patients (FIG. 4b). Importantly, all of the patients with L1 levels greater than 7 ng/ml had advanced stage IV disease.

Discussion

To date, functions attributed to the neural cell adhesion molecule L1 include the potentiation of neural developmental processes such as cerebella cell migration and neurite fasiculation^{24,34}. In this study we present evidence for an expanded and novel role for L1 in the potentiation of angiogenesis. Thus, it is shown that a soluble L1-fragment, that interacts with both αvβ3 and αvβ1, induces a significant angiogenic response in the chick chorioallantoic membrane. Furthermore, we are able to confirm the importance of a single RGD motif in this fragment by demonstrating that a 15-mer L1-peptide containing this RGD motif and the appropriate flanking amino acids also induces a significant angiogenic response. Importantly, we present further evidence that significant amounts of L1 polypeptides are produced by neuroectodermal tumors.

Important mechanistic questions remain with regard to the roles of αvβ3 or αvβ1 as target ligands in the induction of angiogenesis. When considering a potential role for αvβ3, it needs to be recognized that this integrin is either minimally expressed or is absent on quiescent vessels in the 10 day old chick embryo and is only significantly expressed on newly formed angiogenic vessels¹⁰. The integrin avβ1, which has been reported on microvascular endothelial cells³⁵, may therefore be the more relevant target. Our finding that a function blocking antibody to chick β1-integrins but not to αvβ3 is able to induce angiogenesis with a similar dose response profile as the L1-RGD peptide adds some support to the functional importance of αvβ1.

The novel finding that an RGD peptide can induce an angiogenic response needs to be reconciled with the observation that tumor or cytokine induced angiogenesis can be inhibited with a cyclic RGD peptide that interacts with αvβ3^{4,10}. Based on the data presented, it is clear that the L1 RGD peptide only induces angiogenesis over a delineated concentration range, with high concentrations being ineffective. This dose response could be explained if high concentrations of the L1-RGD peptide become inhibitory or anti-angiogenic as described in previous studies with the cyclic RGD-peptide^{4,10}. Thus, a given RGD-peptide may function both as an antagonist and a agonist, depending on concentration and frequency of administration. The induction of angiogenesis by the L1-RGD peptide likely involves an initial interaction with extant quiescent vessels, however, the continued presence of high amounts of RGD peptide could abrogate subsequent events required for vessel development including endothelial cell migration.

While the antagonistic effects of RGD peptides are intuitive, the mechanism by which the L1-RGD peptide or the L1 RGD polypeptide induce neovascularization remains to be defined. Perhaps the most obvious explanation would be perturbation of integrin ligation resulting in a lessening of vascular cell adhesivity. In this regard, Ingber and Folkman³⁶ present evidence that intermediate levels of adhesivity, but not low or high levels, can profoundly potentiate endothelial cell tubulogenesis. This phenomenon has also been shown using anti-integrin antibodies. Thus, Gamble et al.¹² demonstrated that anti-α2β1 and anti-αvβ3 antibodies can potentiate endothelial cell tubologenesis in collagen and fibrin gels respectively. These authors suggest that this phenomenon may result from a reduction in adhesivity or mechanical coupling between cell and matrix, favoring expression of a more motile phenotype and the generation of intra-cellular signaling events secondary to changes in cell shape and/or actin organization. The ability of anti-integrin antibodies to promote in vitro tubologenesis is particularly interesting given our finding that an anti-β1 antibody can induce angiogenesis. These findings also add support to the concept that limited, but not complete, inhibition of adhesion to promote intermediate levels of adhesivity may potentiate angiogenesis.

The extent to which the L1 polypeptide or L1-RGD peptide alters adhesivity between endothelial cells, or endothelial cells and the subendothelial matrix remains to be determined. However, at a high molarity (250 uM) the L1-RGD peptide abrogated 30-40% of HDMEC or ECV304 avb3(−) cell adhesion to fibronectin (data not shown). At lower proangiogenic concentrations (i.e., 25 mM) the L1-RGD peptide did not overtly prevent adhesion to fibronectin but may still have had an effect on the strength of adhesion or on the repertoire of integrins binding. This would certainly be consistent with the fact that both αvβ3 and αvβ1 can promote cell adhesion or spreading on fibronectin³⁷⁻³⁸.

It is conceivable that the induction of angiogenesis by the L1 RGD peptide or by the L1 fusion protein is not due to a single event but is multifactorial. Thus there are numerous examples of RGD-peptides or polypeptides inducing processes that can potentiate angiogenesis. For example, both soluble RGD peptides (4-6 mer) and a soluble RGD-polypeptide fragment (120 kD) derived from fibronectin have been shown to induce expression of matrix metalloproteinases (MMP-1, -2 &-9)¹¹. This is important since dissolution of the subendothelial matrix is an essential early event in the angiogenic process. It is also important to note that RGD peptides have been shown to induce endothelial monolayers or vessel permeability to plasma proteins³⁹. This could be relevant since it has been suggested that microvascular hyperpermeability to plasma proteins is an important and early mechanistic component in the induction of angiogenesis⁴⁰.

Remarkably a number of studies have shown that RGD peptides can function as ‘agonists’ effectively promoting integrin function. Thus several investigators have suggested that short RGD peptides can induce a conformational change in certain integrins resulting in the exposure of otherwise cryptic substrate binding sites. In this regard, Agrez et al.⁴¹ present evidence that suggesting that a tetrameric RGD peptide can induce a conformational change in αv-integrins resulting in the exposure of otherwise cryptic collagen type I binding sites. Significantly as in our findings the agonistic effect of the RGD peptide was only observed over a limited concentration range with higher peptide concentrations being ineffective or inhibitory. Importantly, this effect was also reported to occur with RGD-containing glycoproteins such as vitronectin⁴¹. A comparable phenomenon has been described in the interaction between platelets and polymerizing fibrin. Thus an interaction between platelet aIIbb3 and RGD containing peptides of the fibrinogen a-chain was found to significantly enhance platelet interaction with fibrin resulting in increased clot tension⁴². This again is thought to reflect the ability of RGD containing ligands to induce a conformational change and expose otherwise cryptic ligand-induced binding sites (LIBS). Certain of these LIBS then appear to mediate function⁴³. Another notable ‘agonistic’ function ascribed to RGD peptides is an ability to redistribute both αvβ1 and αvβ3 into focal contacts, despite the fact that these integrins are unable to interact with the ligand supporting adhesion and focal contact formation⁴⁴. It has been suggested that such translocation into adhesion plaques will have important consequences for subsequent signaling events since such plaques are important sites or the initiation of signaling cascades⁴⁵.

While the general relevance of the RGD motif for the induction of angiogenesis has not been established, it is noteworthy that this motif is present in a number of potent angiogenic factors, including, for example, bovine angiogenin and the HIV Tat protein. In both cases these factors have been shown to support integrin-dependent endothelial cell attachment⁴⁶ or tubulogenesis⁴⁷. Numerous studies have documented the ability of RGD-containing ECM components to modulate or even induce angiogenesis. For example, fibronectin has been shown to promote the elongation of microvessels when added to collagen gels⁴⁸ while fibrin is reported to induce angiogenesis in an animal model⁴⁹. However, the extent to which these components are acting as soluble agonists, rather than solid phase ligands is unclear. In both studies the authors discuss the potential role of soluble fibronectin of fibrin degradation products as initiating factors. This then raises the possibility that controlled proteolysis of extracellular matrix could initiate angiogenesis by generating soluble degradation products that contain the RGD motif. This would then provide an interesting parallel to the induction of angiogenesis by proteolytically released L1 RGD polypeptides.

The physiological context of L1 release is vital to understanding its potential role in the induction of angiogenesis. We have provided evidence that significant levels of soluble L1 are released by melanoma or neuroblastoma cell lines strongly suggesting a role in the neovascularization of aggressive neuroectodermal tumors. In this regard, it is interesting that Linnemann et al.²⁸ report finding significant L1 expression on an aggressive metastasizing variant of the melanoma cell line K1735 while no expression was detected on non-metastatic. K1735 cells. Recent reports in which we and others describe L1 on immune cells^{17,29} may also be significant since they suggests that L1 may also be released at sites of inflammation. This is an important observation since it suggests that soluble L1 may be present as a potential angiogenic factor in inflammatory disorders such as rheumatoid arthritis. Finally, it is particularly interesting that increased expression and shedding of L1 has been reported in the context of nerve injury⁵⁰⁻⁵² and that nerve regeneration is associated with both increased capillary formation and vascular permeability⁵³.

In conclusion, we have demonstrated that soluble integrin antagonists can, when applied as a single low dose, induce an angiogenic response. Based this observation, we propose a novel mechanism for the induction of angiogenesis based on the subtle perturbation of integrin binding. We further propose that this mechanism can account for the proangiogenic activity of soluble L1 which is recognized by a variety of vascular integrins¹⁸. The production of soluble L1 by neuroectodermal tumors suggests pathophysiological relevance for tumor neovascularization.

Methods

Reagents and Antibodies. Anti-integrin antibodies used included anti-human and chick αvβ3 MAb LM609 and the anti-chick β1-integrin MAb CSAT. LM609 and CSAT were kindly provided by Dr D. A. Cheresh (The Scripps Research Institute, CA) and Dr C. Buck (Wistar Institute, PA) respectively. An affinity purified anti-human L1 polyclonal antibody and a purified L1 fusion protein consisting of the entire L1 extracellular domain with a 6×His tag (L1-ECD-His) were kindly provided by Dr. W. Stallcup (Burnham Institue, CA).

Peptides. L1 peptides were synthesized on a ABI 430A Peptide Synthesizer within the Scripps Research Core Facility. A 15-mer peptide was selected to include the single RGD site in human L1 (i.e., PSITWRGDGRDLQEL). Control peptides were substituted with alanine resulting in PSITWRADGRDLQEL. For the purpose of immobilization an additional batch of these peptides was synthesized with N-terminal cysteine residues. Peptides were prepared using Rink Amide MBHA or Wang resin (Novabiochem, La Jolla, Calif.). After resin deprotection and assembly the peptides were cleaved from the resin with a cleavage cocktail (2.5% ethanedithiol, 5% thioanisole, 5% water, 87.5% trifluoroacetic acid) and subsequently purified by preparative reverse phase HPLC. Peptides were characterized further by analytical HPLC and mass spectroscopy.

Construction and Expression of L1 Fusion Proteins. The generation and characterization of L1 fusion proteins used in this study has been described in detail³⁰. In brief, three cDNA fragments coding for Ig-like domains 1, 2 and 3 (Ig 1-3), Ig-like domains 4,5, and 6 (Ig 4-6) and for all five fibronectin type-III-like repeats (FN 1-5) were prepared and inserted between the Eco RI and Bam HI sites of pGEX-3×. The cDNA fragment Ig 1-3 codes for amino acids between positions 24 to 351, the cDNA fragment Ig 4-6 codes for amino acids between positions 352 and 595, and the cDNA fragment FN 1-5 codes for amino acids between positions 596 and 1094 (amino acid numbering according to Hlavin and Lemmon, 1991). In all three cases, GST was fused to the amino terminus of the fusion protein. To produce GST-L1 fusion proteins, transformed E. coli., strain JM101, were induced by adding 1 mM isopropyl b-D-thiogalactopyranoside (IPTG) and the induced bacteria subsequently resuspended in a lysis buffer (50 mM Hepes buffer, 5% glycerol, 2 mM EDTA, 0.1 M DTT, pH 7.9). Fusion proteins were isolated from inclusion bodies, solubilized and re-folded as described³⁰. All GST-fusion proteins were subsequently purified by affinity chromatography on a glutathione-Sepharose 4B column and extensively dialyzed against PBS. L1 GST fusion proteins Ig 1-3, Ig 4-6, and FN 1-5 were analyzed by SDS-PAGE and are described by Zhao and Siu³⁰.

Trypsin-treatment of L1-extracellular domain. Purified L1-ECD-His tag fusion protein (700 ml at 1 mg/ml in PBS) was mixed with 250 ml of immobilized trypsin in PBS. The trypsin used was isolated from bovine pancreas and immobilized on crosslinked 4% beaded agarose Pierce, Rockford, Ill.). The slurry was washed repeatedly in PBS prior to the addition of the L1-ECD-His tag fusion protein. Trysinization was performed at room temperature for 15-60 minutes prior to the removal of the immobilized trypsin and analysis by SDS-polyacrylamide gel electrophoresis. Immobilized trypsin was removed by centrifugation and filtration.

Angiogenesis assay. Angiogenesis was assessed using the chick chorioallantoic membrane (CAM) model which has been extensively described⁵⁴. Briefly, ten day old fertilized Leghorn chicken eggs were purchased from Mc Intyre Poultry, San Diego, Calif. The chorioallantoic membrane was dropped away from the shell to create a false air sac and a 1 cm2 window cut in the shell directly above the dropped membrane. Circular filter discs punched out of Whatman 1 filter paper (Whatman, UK) were saturated with approximately 10 ul of fibroblast basal media (FBM; Clonetics, San Diego, Calif.) containing defined concentrations of GST-fusion proteins, peptides or mAbs as indicated in the text. All samples were checked for endotoxin contamination by the Limulus Amebocyte Lysate QCC-1000 assay as described by the manufacturer (BioWhittaker, Walkersville, Mass.) and significantly contaminated preparations excluded. Saturated discs were carefully placed on exposed chorioallantoic membranes avoiding highly vascularized areas. The embryos were subsequently maintained in an incubator at 37° C. and 60% relative humidity for 72 hours prior to harvesting the membrane beneath and adjacent to the disc. Angiogenesis was quantified by counting the number of vessel branch points within the area of the membrane immediately beneath the disc. Counting was performed using an Olympus SZH10 Research Stereo microscope. The number of vessel branch points is proportional to the number of angiogenic blood vessels. Due to the inherent variability of the animal model, treatment groups consisted of a minimum of six embryos, and experiments were performed on three separate occasions by two different investigators.

Enzyme-Linked Immunoabsorbent Assay. Wells of a flexible Falcon 96-well plates (Becton Dickinson, Oxnard, Calif.) were coated overnight at 4° C. with purified anti-L1 MAb 5G3 or with a control murine IgG2a antibody (UPC10: Sigma, St. Louis, Mo.). Both antibodies were offered at 4 ug/ml in PBS. Treated wells were repeatedly washed with a Tris-saline buffer (10 mM Tris, 138 mM NaCl) containing 0.2% Tween-20. Non-specific binding sites were subsequently blocked for 2 hours at 37° C. with 5% BSA in PBS. Different dilutions of serum-free tumor-conditioned media were added to 5G3 Mab or to UPC10 antibody treated wells. Tumor conditioned media was generated by culturing M21 melanoma cells or SK-N-AS cells for 72 hours in RPMI-1640 and 1% glutamine alone. Cells were removed by centifugation and cell number per ml of media determined. Samples of tumor-conditioned media were diluted in a tris-saline dilution buffer (10 mM Tris, 138 mM NaCl) containing 0.2% Tween-20 and 0.1% BSA and were added to the wells for 2 hours at room temperature. In further experiments, serum samples from normal individuals or neuroblastoma patients (stage I-IV) were diluted 1:10 in PBS containing 0.2% Tween-20 and were also added to the antibody treated wells for 2 hours at room temperature. After a series of washes, the wells were incubated for 90 minutes with an affinity purified anti-L1 rabbit polyclonal antibody diluted to 2.5 ug/ml in a Tris-saline buffer (10 mM Tris, 138 mM NaCl) containing 0.2% Tween-20. Bound rabbit antibody was detected using a human absorbed goat anti-rabbit IgG-horseradish peroxidase conjugate (Southern Biotechnology Associates, Birmingham, Ala.) diluted 1 in 6000 in the tris-saline dilution buffer. Color was developed by the addition of 100 ul of 0.4 mg/ml 0 phenylenediamine dihydrochloride (Sigma, St. Louis, Mo.) and 0.014% hydrogen peroxide in a 0.05M phosphate-cirate buffer, pH 5. Plates were read at 450 nm on a microplate reader (Kinetic Microplate Reader, Molecular Devices, Sunnyvale, Calif.). Absolute amounts of L1 were determined by reference to a standard curve obtained by addition and titration of a purified L1-His fusion protein (2-150 ng/ml) containing the entire extracellular domain of L1.

### REFERENCES

1. Werb, Z. et al. Signal transduction through the fibronectin receptor induces collagenase and stromelysin gene expression. J. Cell Biol. 109, 877-889 (1989).

2. Brooks, P. C. et al. Localization of matrix metalloproteinase MMP-2 to the surface of invasive cells by interaction with integrin alpha-v beta-3. Cell 85, 683-693 (1996).

3. Meredith, J. E. et al. The extracellular matrix as a survival factor. Mol. Biol. Cell 4, 953-961 (1993).

4. Brooks, P. C. et al. Integrin avb3 antagonists promote tumor regression by inducing apoptosis of angiogenic blood vessels. Cell 79, 1157-1164 (1994).

5. Sage, E. H. et al. Pieces of eight: bioactive fragments of extracellular proteins as regulators of angioegnesis. Trends Cell Biol. 7, 182-186 (1997).

6. Leavesley D. I. et al. Integrin b1- and b3-mediated endothelial cell migration is triggered through distinct signaling mechanisms. J. Cell Biol. 121, 163-170 (1993).

7. Bischoff, J. Approaches to studying cell adhesion molecules in angiogenesis. Trends Cell Biol. 5, 69-74 (1995).

8. Luscinskas, F. W. and J. Lawler. Integrins as dynamic regulators of vascular function. FASEB J. 8, 929-938 (1994).

9. Drake, C. J. et al. Antibodies to beta 1-integrins cause alterations of aortic vasculogenesis in vivo. Dev. Dynamics 193, 83-91 (1992).

10. Brooks, P. C. et al. Requirement of vascular integrin alpha-v beta-3 for angiogenesis. Science 264, 569-571 (1994).

11. Arner, E. C. and M. D. Tortorella. Signal transduction through chondrocyte integrin receptors induces matrix metalloproteinase synthesis and synergizes with interleukin-1. Arthritis Rheumatism 38, 1304-1314 (1995)

12. Gamble, J. R. et al. Regulation of in vitro capillary tube formation by anti-integrin antibodies. J. Cell Biol. 121, 931-943 (1991).

13. Matsumura, T., K. et al. Endothelial cell tube formation depends on cadherin 5 and CD31 interactions with filamentous actin. J. Immunol. 158, 3408-3416 (1997).

14. Koch, A. E. et al. Angiogenesis mediated by soluble forms of E-selectin and vascular cell adhesion molecule-1. Nature 376, 517-519 (1995).

15. Polverini, P. J. Cellular adhesion molecules. Newly identified mediators of angiogenesis. Am. J. Pathol. 148, 1023-1029 (1996).

16. Montgomery, A. M. P. et al. Human neural cell adhesion molecule L1 and rat homologue NILE are ligands for integrin avb3. J. Cell Biol. 132, 475-485 (1996).

17. Ebeling, O. et al. L1 adhesion molecule on human lymphocytes and monocytes: expression and involvement in binding to avb3 integrin. Eur. J. Immunol. 26, 2508-2516 (1996).

18. Felding-Habermann B. et al. A single immunoglobulin-like domain of the human neural cell adhesion molecule L1 supports adhesion by multiple vascular and platelet integrins. J. Cell Biol.139, 1567-1581 (1997).

19. Moos, M. et al. Neural adhesion molecule L1 is a member of the immunoglobulin superfamily with binding domains similar to fibronectin. Nature. 334, 701-703 (1988).

20. Sonderegger, P. R., and F. G. Rathjen. Regulation of axonal growth in the vertebrate nervous system by interactions between glycoproteins belonging to two subgroups of the immunoglobulin superfamily. J. Cell Biol. 119, 1387-1394 (1992).

21. Hlavin, M. L., and V. Lemmon. Molecular and functional testing of human L1CAM: An interspecies comparison. Genomics 11, 416-423 (1991).

22. Faissner, A. et al. Biosynthesis and membrane topography of the neural cell adhesion molecule L1. EMBO (Eur. Mol. Biol. Organ.). 4, 3105-13 (1985).

23. Sadoul, K., R. et al. Biochemical characterization of different molecular forms of the neural cell adhesion molecule L1. J. Neurochem. 50, 510-21 (1988).

24. Lindner, J. et al. L1 mono- and polyclonal antibodies modify cell migration in early postnatal mouse cerebellum. Nature. 305, 427-430 (1983).

25. Rathjen, F. G., and M. Schachner. Immunocytological and biochemical characterization of a new neuronal cell surface component (L1 antigen) which is involved in cell adhesion. EMBO (Eur. Mol. Biol. Organ.). 3, 1-10 (1984).

26. Reid, R. A., and J. J. Hemperly. Variants of human L1 cell adhesion molecule arise through alternate splicing of RNA. J. Mol. Neurosci. 3, 127-135 (1992).

27. Mujoo, K. et al. Characterization of a unique glycoprotein antigen expressed on the surface of human neuroblastoma cells. J. Biol. Chem. 261, 10299-10309 (1986).

28. Linnemann, D. et al. Differential expression of cell adhesion molecules in variants of the K1735 melanoma cells differing in metastatic capacity. Int. J. Cancer. 43, 709-712 (1989).

29. Pancook, J. D. et al. Expression and regulation of the neural cell adhesion molecule L1 on human cells of myelomonocytic and lymphoid origin. J. Immunol.158, 4413-4421 (1997).

30. Zhao, X., and Siu, C-H, Co-localization of the homophilic binding site and the neuritogenic activity of the cell adhesion molecule L1 to the second Ig-like domain. J. Biol. Chem. 270, 29413 29421 (1995).

31. Nybroe O. et al. A developmental study of soluble L1. Int. J. Dev. Neurosci. 8, 273-281 (1990).

32. Poltorak M. et al. Disturbances in the cell recognition molecules (NCAM and L1) in the CSF of patients with schizophrenia. Exp. Neurol. 131, 266-272 (1995).

33. Liljelund, P. et al. Expression of the neural axon adhesion molecule L1 in the developing and adult rat brain. J. Biol. Chem. 269, 32886-32895 (1994).

34. Lemmon, V. et al. L1-mediated axon outgrowth occurs via a homophilic binding mechanism. Neuron. 2, 1597-1603 ( ).

35. Klein, S. et al. Basic fibroblast growth factor modulates integrin expression in microvascular endothelial cells. Mol. Biol. Cell 4, 973-982 (1993).

36. Ingber, D. E. and J. Folkman. Mechanochemical switching between growth and differentiation during fibroblast growth factor-stimulated angiogenesis in vitro: role of extracellular matrix. J. Cell Biol. 109, 317-330 (1989).

37. Zhang, Z. et al. The avb1 integrin functions as a fibronectin receptor but does not support fibronectin matrix assembly and cell migration on fibronectin. J. Cell Biol. 122, 235-242 (1993).

38. Marshall, J. F. et al. avb1 is a receptor for vitronectin and fibrinogen, and acts with a5b1 to mediate spreading on fibronectin. J. Cell Sci. 108, 1227-1238 (1995).

39. Curtis, T. M. et al. Fibronectin attenuates increased endothelial cell monolayer permeability after RGD-peptide, anti-alpha 5 beta 1, or TNF-alpha exposure. Am. J. Physiol. 269, L248-260 (1995).

40. Dvorak, H. F. et al. Vascular permeability factor/vascular endothelial growth factor, microvascular hyperpermeability, and angiogenesis. Am. J. Pathol. 146, 1029-1039 (1995).

41. Agrez, M. V. et al. Arg-Gly-Asp-containing peptides expose novel collagen receptors on fibroblasts; implications for wound healing. Cell Regulation 2, 1035-1044 (1991).

42. Cohen, I. et al. The effect of peptides and monoclonal antibodies that bind to platelet glycoprotein IIb-IIIa complex on the development of clot tension. Blood 73, 1880-1887 (1989).

43. Frelinger, A. L. et al. Selective inhibition of integrin function by antibodies specific for ligand occupied conformers. J. Biol. Chem. 265, 6346-6252 (1990).

44. LaFlamme, S. E. et al. Regulation of fibronectin receptor distribution. J. Cell Biol. 117, 437-447 (1992).

45. Koivunen E., B. et al. Peptides in Cell Adhesion Research. Methods Enzymol. 245, 346-369 (1994).

46. Soncin F. Angiogenin supports endothelial and fibroblast cell adhesion. Proc. Natl. Acad. Sci. USA. 89,2232-2236 (1992).

47. Albini, A., G. et al. Angiogenic properties of human immunodeficiency virus type 1 Tat protein. Proc. Natl. Acad. Sci. USA. 92, 4838-4842 (1995).

48. Nicosia, R. F. et al. Fibronectin promotes the elongation of microvessels during angiogenesis in vitro. J. Cell. Physiol. 154, 654-661 (1993).

49. Dvorak, H. F. et al. Fibrin containing gels induce angiogenesis. Implications for tumor stroma generation and wound healing. Lab. Invest. 57, 673-686 (1995).

50. Blaugrund, E. et al. Immunological evidence that the neural adhesion molecule L1 is expressed in fish brain and optic nerve: possible association with optic nerve regeneration. Brain Res. 530, 239-44-(1990).

51. Poltorak, M. et al. Degradation fragments of L1 antigen enhance tyrosine hydroxylase immunoreactive neurite outgrowth in mesencephalic cell culture. Brain Res. 619, 255-62 (1993).

52. Tacke, R. and R. Martini. Changes in expression of mRNA specific for cell adhesion molecules (L1 and NCAM) in the transected peripheral nerve of the adult rat. Neuroscience Letters 120, 227 30 (1990).

53. Hobson, M. I. et al. Inter-relationship between angiogenesis and nerve regeneration: a histochemical study. British J. Plastic Surgery 50, 125-131 (1997).

54. Vu, M. et al. Methods in laboratory investigation: An evaluation of methods to quantitate the chick chorioallantoic membrane assay in angiogenesis. Lab Invest. 53, 499-508 (1985).

Since modifications will be apparent to those of skill in this art, it is intended that this invention be limited only by the scope of the appended claims.

1. A method for enhancing angiogenesis, which method comprises administering an effective amount of an integrin binding pro-angiogenic agent to a mammal, wherein angiogenesis is desirable, thereby enhancing angiogenesis in said mammal.

2. The method of claim 1, wherein the integrin binding pro-angiogenic agent is a protein, a polypeptide or a peptide.

3. The method of claim 2, wherein the protein, polypeptide or peptide pro-angiogenic agent binds to an integrin containing the β1 subunit.

4. The method of claim 2, wherein the protein, polypeptide or peptide pro-angiogenic agent contains an integrin binding sequence.

5. The method of claim 4, wherein the integrin binding sequence contains the RGD motif, a RGD related motif or a non-RGD integrin recognition motif.

6. The method of claim 1, wherein the integrin binding pro-angiogenic agent is a small molecule agent.

7. The method of claim 1, wherein the integrin binding pro-angiogenic agent is an anti-integrin antibody, or a derivative or fragment thereof.

8. The method of claim 7, wherein the anti-integrin antibody is a monoclonal antibody.

9. The method of claim 7, wherein the anti-integrin antibody is an antibody against an integrin containing the β1 subunit.

10. The method of claim 9, wherein the anti-β1 function-blocking antibody is selected from the group consisting of CSAT, AG89, QE.2E5, mAb 13 and NaM160-1A3.

11. The method of claim 1, wherein the pro-angiogenic agent perturbs integrin ligation resulting in a lessening of vascular cell adhesivity.

12. The method of claim 1, wherein the pro-angiogenic agent induces a conformational change in the integrin and exposes otherwise cryptic ligand-induced binding sites (LIBS).

13. The method of claim 12, wherein the LIBS is a cryptic collagen type I binding site.

14. The method of claim 1, wherein the pro-angiogenic agent redistributes the integrin into focal contacts.

15. The method of claim 1, wherein the pro-angiogenic agent promotes vascular cell migration and/or protease activity.

16. The method of claim 1, wherein the pro-angiogenic agent is a neural cell adhesion molecule L1 (NCAM L1) or a functional derivative or fragment thereof that substantially retains its binding affinity with the integrin, or a nucleic acid encoding said NCAM L1 or functional derivative or fragment thereof.

17. The method of claim 16, wherein the NCAM L1 is a soluble NCAM L1 or a functional derivative or fragment thereof that substantially retains its binding affinity with the integrin.

18. The method of claim 16, wherein the NCAM L1 comprises the entire extracellular domain of the NCAM L1 or a functional derivative or fragment thereof that substantially retains its binding affinity with the integrin.

19. The method of claim 16, wherein the NCAM L1 comprises the Ig-like domains 4-6 (Ig 4-6) of the extracellular domain of the NCAM L1 or a functional derivative or fragment thereof that substantially retains its binding affinity with the integrin.

20. The method of claim 1, wherein the pro-angiogenic agent is a protein or a peptide that specifically binds to an antibody that is raised against a peptide having the following amino acid sequence:
PSITWRGDGRDLQEL.

21. The method of claim 20, wherein the peptide has the following amino acid sequence:
PSITWRGDGRDLQEL.

22. The method of claim 1, wherein the integrin contains a β1 subunit.

23. The method of claim 1, wherein the integrin comprises α5β1 or αvβ1 subunits.

24. The method of claim 1, wherein the mammal is a human.

25. The method of claim 1, wherein the mammal has an ischemic disease or wound healing disorder.

26. An isolated protein or peptide, which protein or peptide is selected from the group consisting of the entire extracellular domain of the NCAM L1 or a functional derivative or fragment thereof that substantially retains its binding affinity with the integrin, a NCAM L1 comprising the Ig-like domains 4-6 (Ig 4-6) or a functional derivative or fragment thereof that substantially retains its binding affinity with the integrin, a protein or a peptide that specifically binds to an antibody that is raised against a peptide having the following amino acid sequence: PSITWRGDGRDLQEL and a peptide having the following amino acid sequence: PSITWRGDGRDLQEL.

27. A pharmaceutical composition, which composition comprises an isolated protein or peptide of claim 26 and a pharmaceutically acceptable carrier or excipient.

28. An isolated nucleic acid, which nucleic acid encodes a protein or peptide selected from the group consisting of the entire extracellular domain of the NCAM L1 or a functional derivative or fragment thereof that substantially retains its binding affinity with the integrin, a NCAM L1 comprising the Ig-like domains 4-6 (Ig 4-6) or a functional derivative or fragment thereof that substantially retains its binding affinity with the integrin, a protein or a peptide that specifically binds to an antibody that is raised against a peptide having the following amino acid sequence: PSITWRGDGRDLQEL and a peptide having the following amino acid sequence: PSITWRGDGRDLQEL.

29. A pharmaceutical composition, which composition comprises a nucleic acid of claim 28 and a pharmaceutically acceptable carrier or excipient.

30. A combination, which combination comprises:
a) an effective amount of an integrin binding pro-angiogenic agent; and
b) an effective amount of another angiogenic molecule.

31. The combination of claim 30, which is in the form of a pharmaceutical composition.

32. The combination of claim 30, wherein the other angiogenic molecule is an angiogenic cytokine, or a functional derivative or fragment thereof that substantially retains its angiogenic activity, or a nucleic acid encoding an angiogenic cytokine, or a functional derivative or fragment thereof that substantially retains its angiogenic activity.

33. The combination of claim 32, wherein the angiogenic cytokine is selected from the group consisting of an acidic fibroblast growth factor (aFGF), an angiopoietin, a basic fibroblast growth factor (bFGF), a heparin-binding epidermal growth factor (HB-EGF), an insulin-like growth factor (IGF), a placental growth factor (PIGF), a platelet derived growth factor (PDGF), a scatter factor hepatocyte growth factor (HGF), a transforming growth factor-beta (TGF-beta) and a vascular endothelial growth factor (VEGF).

34. A method for enhancing angiogenesis, which method comprises administering an effective amount of a combination of claim 30 to a mammal, wherein angiogenesis is desirable, thereby enhancing angiogenesis in said mammal.

35. A method for enhancing angiogenesis, which method comprises administering an effective amount of an integrin antagonist to a mammal, wherein angiogenesis is desirable, thereby enhancing angiogenesis in said mammal.

36. The method of claim 35, wherein the integrin antagonist is selected from the group consisting of an integrin anti-sense oligonucleotide, an anti-integrin antibody, a soluble integrin, or a derivative or fragment thereof, and an agent that reduces or inhibits production of the integrin.

37. A combination, which combination comprises:
a) an effective amount of an integrin antagonist; and
b) an effective amount of another angiogenic molecule.
